(19) United States
(12) Patent Application Publication
(10) Pub. No.: US 2008/0125771 A1
(43) Pub. Date: May 29, 2008

(54) METHODS AND APPARATUSES FOR CONTOURING TISSUE BY SELECTIVE APPLICATION OF ENERGY

(76) Inventors: Michael Lau, Edmonds, WA (US); Leonard F. Pease, Richland, WA (US)

Correspondence Address: PERKINS COIE LLP;PATENT-SEA, P.O. BOX 1247, SEATTLE, WA, 98111-1247, US

(21) Appl. No.: 11/945,791

(22) Filed: Nov. 27, 2007

Related U.S. Application Data
(60) Provisional application No. 60/861,314, filed on Nov. 27, 2006.

(51) Int. Cl. A61B 18/00

(52) U.S. Cl. 606/41; 606/27

(57) ABSTRACT

Methods and apparatuses for contouring, shaping and/or directionally shrinking tissue by selective application of energy are disclosed herein. One embodiment of a method of contouring tissue includes determining a contraction direction along which the tissue is to be preferentially contracted and applying energy to a plurality of discrete elongated exposed regions of tissue spaced apart from each other among non-exposed regions of tissue. At least one of the exposed regions can be oriented such that a longitudinal dimension of the exposed regions is generally transverse to the contraction direction.

### CROSS-REFERENCE TO RELATED APPLICATION

This application claims the benefit of U.S. Provisional Application No. 60/861,314, filed on Nov. 27, 2006, entitled “METHODS FOR THE THERAPEUTIC CONTRACTION AND/OR SHAPING OF COLLAGENOUS TISSUES BY SELECTIVE DIRECTIONAL APPLICATION OF ENERGY,” the entirety of which is incorporated by reference herein.

### BACKGROUND

Traditional cosmetic, plastic and orthopedic surgeries cut, trim, suture and cauterize the target tissue. Although these traditional techniques shape and contour the target tissue, they are invasive, risk infection, require extensive recovery and increase morbidity. To avoid these disadvantages, minimally invasive or non-invasive approaches are frequently used to treat the target tissue. For example, applying energy to collagen containing tissues can achieve the shrinkage or contraction necessary to resolve a variety of medical conditions, such as urinary incontinence, joint laxity, shoulder instability and the superficial effects of aging. Previous non-invasive approaches, however, fail to shape and contour the target tissue with specific control or directionality that is generally desired.

Clinical and investigative approaches to shrinking tissue containing collagen include exposing the tissue to alternating current in the radio frequency range through small probes (Medvecky, 2001), laser irradiation (Vangsness, 1997; Xiao, 2006), ultrasound (Brown, 2005), and hot water (Wall, 1998). These methods generally disrupt collagen's triple helix within the tissue directly by targeting specific bond energies or indirectly by heating the surrounding materials to unwind the strands of the helix. In either case, these methods work by increasing the entropic contribution to the free energy of the exposed tissue, thus overcoming the enthalpic contributions that hold the collagen together. Accordingly, any source of thermal or vibrational energy may disturb the collagen matrix as the application of thermal energy transforms the tertiary structure of ordered and aligned collagen (the long, rod-like triple helix) into random, bulbous coils. This causes a net decrease in the length of the tissue along the original axis of the aligned fibrous collagen. Thus, in tissues with oriented fibers (e.g., tendons, fascia, ligaments, etc.) shrinkage will take place along the axis of orientation of the fibers. However in tissues with less orientation (e.g., skin, cornea, etc.), the shrinkage will be less directional (Hersh, 2005).

In several conventional tissue shrinkage applications, irrespective of energy modality (e.g., RF, high intensity focused ultrasound or HIFU, microwave, electromagnetic energy, direct thermal heating, etc.), the energy is generally applied to a target area in an arbitrary and capricious manner, thereby shrinking the tissue non-directionally. The resulting shrinkage is accordingly unpredictable in its character, shape and/or durability (i.e., non-directional). For example, conventional tissue shrinking applications such as RF treatment of paravaginal tissues to correct stress incontinence do not always yield predictable and repeatable results. In addition, other applications including forms of energy different from RF, such as plasma or laser treatment of skin and subcutaneous tissue, are generally applied to the target tissue with no directionality or stress-strain control of the target tissue.

Specific attempts have been made to conform and contour the skin surface by applying mechanical force to the bodily structure while applying electromagnetic energy (e.g., Knowlton, U.S. Pat. No. 6,470,216). This approach depends on the temporary conformation of the tissue to a conformer (e.g., a mold) during energy application. However, the tissue may return to its original shape after releasing the mechanical force because there is no demonstrable, intrinsic change to the tissue itself. Another approach seeks to stimulate collagen production by delivering HIFU in a manner that creates lesions for the purpose of skin rejuvenation (e.g., Hissong et al., U.S. Pat. No. 6,595,934). Hissong et al., however, does not disclose forming lesions that directionally contour the target tissue. Another approach, termed Fractional Photothermolysis, forms arrays of microscopic columns of ablated thermal injury by laser irradiation to treat facial rhytides (e.g., Geronemus, 2006). This approach intends to facilitate more rapid healing and tissue repair by reducing the distance for migration from the non-exposed tissue surrounding the ablated columns. Yet again, however, this approach does not directionally contour the target tissue.

Several conventional tissue shrinking applications are challenging because the target tissue may lose its mechanical integrity. For example, applying energy with a sharp probe deposits a substantial amount of energy to a very small area. (Medvecky, 2001). This can lead to charring of the tissue, which increases the likelihood of mechanical failure upon subsequent stressing of the tissue. Similarly, applying energy indiscriminately over a broad target region can also lead to undesirable consequences (Medvecky, 2001). Shrinkage or contraction of collagen results in an immediate change in the elastic modulus or stiffness of the tissue (Wall, 1998). The cyclic stresses of fatigue also affect the elastic modulus (Wren, 2003). Accordingly, applying energy indiscriminately or capriciously may result in either increased droopiness or loss of mechanical integrity (e.g., by affecting the modulus of elasticity). Either result decreases the efficacy and duration of surgical intervention.

### BRIEF DESCRIPTION OF THE FIGURES

FIG. 1 is a schematic flow diagram of a process for contouring tissue in accordance with an embodiment of the disclosure.

FIG. 2A is a front view of a breast, FIG. 2B is a side view of the breast, and FIG. 2C is a front view of the breast with a plurality of exposed regions of tissue in accordance with a further embodiment of the disclosure.

FIGS. 3A and 3B are schematic diagrams of patterns of exposed regions of tissue within a contour zone in accordance with embodiments of the disclosure.

FIGS. 4A and 4B are schematic diagrams of a contour zone before and after non-selective energy exposure, respectively, in accordance with an embodiment of the disclosure.

FIGS. 5A-5D are schematic diagrams of a contour zone illustrating the effect of selective energy application to a portion of the contour zone in accordance with a further embodiment of the disclosure.

FIGS. 6A-6D are schematic diagrams of contour zones in accordance with other embodiments of the disclosure.

FIGS. 7A-7D are schematic diagrams of contour zones having an induced curvature due to varying depths of applied energy in accordance with still another embodiment of the disclosure.

FIG. 8A is a schematic top view of a contour zone and FIG. 8B is a schematic isometric view of the contour zone having energy applied to different depths of the contour zone in accordance with another embodiment of the disclosure.

FIGS. 9-12 are isometric views of apparatuses in accordance with several embodiments of the disclosure for facilitating energy exposure.

FIG. 13 is a schematic diagram of a system for contouring tissue in accordance with embodiments of the disclosure.

### DETAILED DESCRIPTION

### A. Overview

The following disclosure describes several methods and apparatuses for contouring or selectively shaping tissue. The embodiments described below contour tissue in predetermined or desired directions by delivering energy to discrete exposed regions of tissue within one or more contour zones in and/or on the body. In certain embodiments, the exposed regions have shapes and are arranged in patterns that cause the tissue to contract in one or more selected directions. The exposed regions can be elongated, e.g., they can have an aspect ratio greater than unity. In certain embodiments, for example, the exposed regions can include one or more rectangular geometries oriented such that the longitudinal dimension of at least one of the exposed regions is oriented to induce more contraction of the tissue in one direction than another. In other embodiments, the exposed regions can include shapes such as ellipses, ovals and/or other polygonal shapes with or without rounded corners. In still further embodiments, energy may be applied to exposed regions of tissue, to at least partially induce auxetic characteristics to the tissue.

Target areas or contour zones having one or more exposed regions can have different patterns and densities to contract the tissue with controlled directionality. For example, in some embodiments the exposed regions may be non-uniformly distributed in the contour zone. In other embodiments, the density of the exposed areas may differ across the contour zone. Devices for delivering the energy to the skin can be configured to deliver energy within the body transcutaneously, transluminally and/or through incisions. The energy can be of any modality including, without limitation, infrared or other heat, radiofrequency, microwave, light and/or ultrasound including high intensity focused ultrasound (HIFU). The apparatus and methods disclosed herein can be used for any application, including for example, cosmetic applications (e.g., mastopexy, wrinkles, etc.), urinary incontinence, joint laxity, joint stability, organ treatments and/or other suitable applications. In addition, tissue imaging techniques can be used in conjunction with the energy application. For example, the imaging techniques can include x-ray, ultrasound, CT scan, positron emission topography (PET), MRI, etc.

According to an embodiment of the disclosure, a method for contouring tissue includes determining a contraction direction along which the tissue is to be contracted to a greater extent relative to other directions. The method also includes applying energy to a plurality of discrete elongated exposed regions of tissue. The exposed regions of tissue are spaced apart from each other among non-exposed regions of tissue. In addition, the exposed regions can be oriented such that a longitudinal dimension of at least one of the exposed regions is generally transverse to the contraction direction.

Another embodiment is directed to a method for contouring tissue, including determining an arrangement of at least one contour zone of tissue and selectively applying energy to one or more discrete portions of the tissue in the contour zone. Selectively applying the energy contracts at least a portion of the tissue in a predetermined direction to a greater extent relative to directions at an angle to the desired direction.

An apparatus for shaping tissue configured in accordance with an embodiment of the disclosure includes a support member and an energy applicator coupled to the support member. The applicator is configured to apply energy to an area of the tissue to contract the tissue in a predetermined direction. An apparatus for facilitating tissue contouring according to another embodiment of the disclosure includes a body configured to be positioned proximate to the tissue and at least one exposure area in the body. The exposure area has a longitudinal dimension and a lateral dimension, with the longitudinal dimension being greater than the lateral dimension. The exposure area exposes a target area of the tissue such that energy can be applied through the exposure area to the target area. In certain embodiments, the apparatus can include a support member attached to the body and configured to allow a user to reposition the body proximate to the tissue. The apparatus can also be used to selectively cool portions of the tissue. For example, in one embodiment, the apparatus can be configured to thermoelectrically cool portions of the tissue. In other embodiments, the apparatus can include at least one channel extending through the body through which a coolant can be disposed. The channel can extend through a portion of the body that is proximate to the exposure area.

A system for contouring tissue configured in accordance with a further embodiment of the disclosure includes a computer readable medium operably coupled to an energy source. The system also includes an energy applicator operably coupled to the energy source. The computer readable medium contains instructions that cause the applicator to selectively apply energy to shrink at least a portion of the tissue in a predetermined direction. In certain embodiments, the system can also include an imaging system operably coupled to the computer readable medium and configured to produce an image of at least a portion of a contour zone of the tissue.

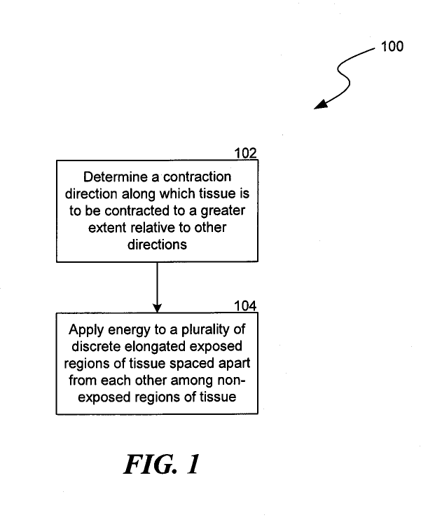

Specific details of several embodiments of the disclosure are set forth in the following description and FIGS. 1-13 to provide a thorough understanding of these embodiments. A person skilled in the art, however, will understand that the disclosure may be practiced without several of these details or additional details can be added to the disclosure. Moreover, several details describing well-known structures or processes often associated with tissue shrinking devices and methods are not shown or described below in detail to avoid unnecessarily obscuring the description of the embodiments of the disclosure. Where the context permits, singular or plural terms may also include the plural or singular term, respectively. Furthermore, the term “comprising” is used throughout to mean including at least the recited feature(s) such that any greater number of the same feature or additional types of features are not precluded.

### B. Embodiments of Tissue Contouring Methods and Associated Principles

FIG. 1 is a flow diagram illustrating a process 100 for contouring tissue by applying energy to select portions of tissue to contract the tissue in a desired or predetermined direction. The contouring process 100 includes determining a contraction direction along which the tissue is to be contracted to a greater extent relative to other directions (block 102). In certain embodiments, the contraction direction can be generally parallel to a stress applied to the tissue, such as gravity. For example, the ptosis of the breast results from gravity pulling the breast down while a person is in an erect position, thereby lengthening the fascial tissue above the main mass of the breast. As such, a desired contraction direction can be a direction that is generally parallel to gravity when the body is in an erect position. In other embodiments, however, the contraction direction can include other directions relative to an applied stress, including directions generally normal to the applied stress.

The contouring process 100 further includes applying energy to a plurality of discrete elongated exposed regions of tissue spaced apart from each other among non-exposed regions of tissue (block 104). The exposed regions are oriented such that a longitudinal dimension of at least one of the exposed regions is generally transverse to the contraction direction. As described below, the exposed regions can be arranged in different patterns on the tissue. Moreover, the energy applied to the tissue can include various modalities, including but not limited to, electromagnetic waves (e.g., infrared, visible light, ultraviolet, etc.), radio frequency current, microwave, laser, maser, ultrasound, a direct heat source (e.g., a heated liquid, solid or gas), plasma and/or any other suitable energy source for tissue contouring. The application of the energy can be focused, such as HIFU, laser, focused microwave, focused light, etc., or the applied energy may be applied diffusely. The energy can also be applied directly by contact with the target tissue, or the energy can be applied through a content medium to be focused at different depths within the tissue away from the contact surface. In addition, the energy can be simultaneously applied to a plurality of exposed regions, or sequentially applied to the exposed regions.

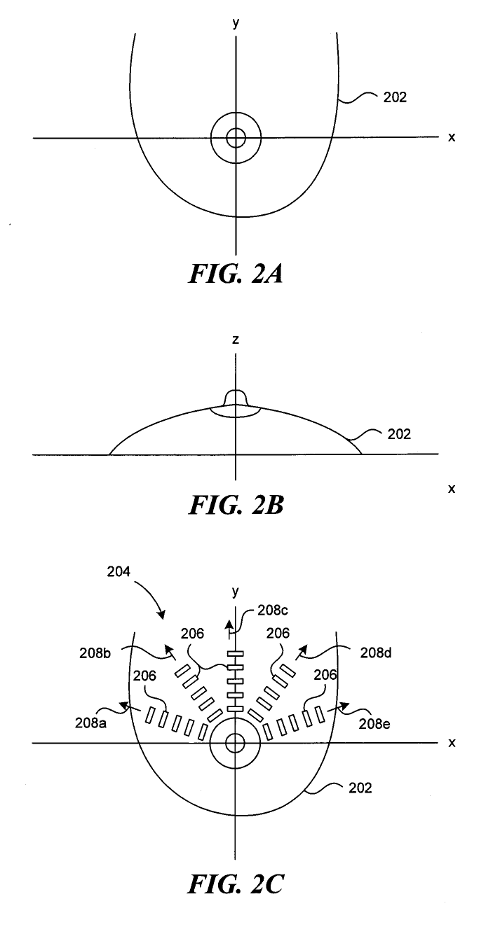

One application of contouring tissue according to an embodiment of the disclosure is directed to a mastopexy (i.e., a breast lift), as described with reference to FIGS. 2A-2C. FIG. 2A, more specifically, is a front view of a breast 202 with reference to an x-y grid, and FIG. 2B is a side view of the breast 202 with reference to an x-z grid. In FIGS. 2A-2C, as well as in other Figures described herein, for purposes of illustration the y direction generally corresponds to the direction of the force of gravity. Referring to FIGS. 2A and 2B together, elongation of the fascia of the breast 202 occurs mostly along the y axis. As the breast tissue (most of which is fatty and fibrous) becomes displaced along the y axis, the breast tissue also becomes redistributed along the x axis as well as along the z axis.

FIG. 2C is a front view of the breast 202 illustrating a pattern 204 of individual exposed regions 206 of tissue in accordance with an embodiment of the disclosure. Energy can be applied to each of the exposed regions 206 of the tissue to selectively contour the breast 202 in one or more directions. More specifically, arrows generally indicate desired contraction directions 208 (identified individually as contraction directions 208a-208e) for different areas of the breast 202. The illustrated contraction directions 208 are radiating generally outwardly from a center portion of the breast 202. Accordingly, the overall contour effect will be to lift portions of the breast 202 in a direction generally parallel to the force of gravity, as well as in directions at other angles relative to the force of gravity.

In the illustrated embodiment, the exposed regions 206 are positioned in columns aligned with the respective contraction directions 208. The exposed regions 206 are also positioned such that their longitudinal dimensions are generally transverse to the respective contraction direction 208. In other embodiments, however, and as described below, the exposed regions 204 can be arranged in different patterns and/or orientations to achieve a desired contouring effect (e.g., staggered or random patterns). The exposed regions 204 can also have different geometries or shapes to achieve the desired contouring effect. In even further embodiments, a single exposed region 206 can be used, rather than a plurality of exposed regions 206. The resulting shaping effect will be the lifting and contouring of the fascia and support tissue of the breast 202, along with the fatty breast tissue attached to the fascia and supporting tissue. Different energy modalities, as described above, can be applied to the exposed regions 206 to induce the net directionally of the breast tissue.

The procedure illustrated in FIGS. 2A-2C achieves a breast lift having results comparable to those of open surgery. The embodiments disclosed herein, however, avoid the complicated and invasive steps associated with open surgery for conventional breast lifts. For example, the disclosed tissue contouring is achieved without cutting or suturing the target tissue, thus reducing or eliminating the chance of scarring and infection. The disclosed methods also do not require a long recovery time associated with conventional breast lifts as incisions do not have to heal.

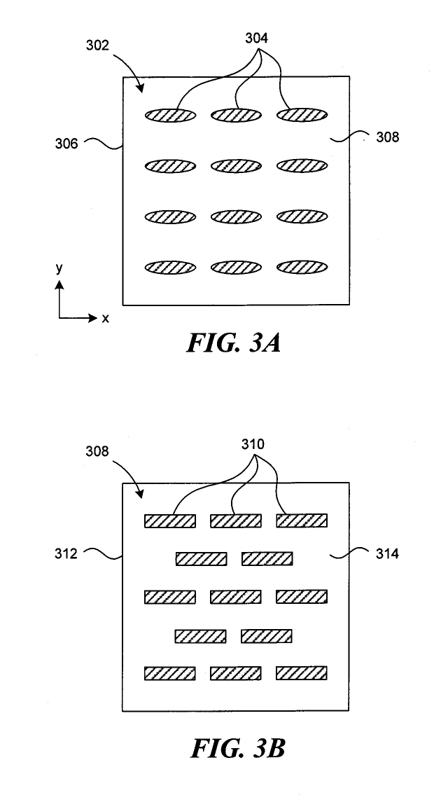

According to several embodiments of the disclosure, the individual exposed regions of tissue can have different shapes and be arranged in different configurations. FIGS. 3A and 3B, for example, are schematic diagrams illustrating patterns of exposed regions of tissue with reference to an x-y grid. In FIGS. 3A and 3B, the y direction generally corresponds to a desired contraction direction. FIG. 3A includes a first pattern 302 of individually exposed regions 304 of tissue in a first contour zone 306. In the first pattern 302, the individually exposed regions 304 have elliptical shapes and are generally aligned in generally parallel columns in the y-direction. The exposed regions 304 are spaced apart from each other and interspersed among the non-exposed tissue 308 in the contour zone 306. FIG. 3B illustrates a second pattern 308 of exposed regions 310 interspersed among non-exposed tissue 314 of a second contour zone 312. The exposed regions 310 of the second pattern 308 are generally arranged in a checkered or staggered pattern. In other embodiments, the exposed regions 304, 310 may be arranged in other patterns, including patterns that are symmetrical, staggered, randomly oriented, columns at diverging angles, etc. Moreover, the density of the exposed regions 310 within a contour zone 312 can also vary according to the parameters of the tissue contouring, including, for example, the tissue type, tissue location, shape of the contour zone, pattern and shape of individual exposed regions, etc.

The geometry or shape of the individual exposed regions 304, 310 affects the contouring of the tissue. For example, in certain embodiments, the exposed regions are elongated, meaning that the aspect ratio (i.e., the ratio of the long dimension to the short dimension) exceeds unity. The exposed regions 304, 310 described above can have characteristic lengths (e.g., the length of the long dimension) ranging from 10 microns to 25 centimeters. In addition, the geometry of the exposed regions can include ellipses, ovals, rectangles, rectangles with rounded corners, isosceles triangles, etc. Moreover, in other embodiments the exposed regions can be configured not to include sharp corners.

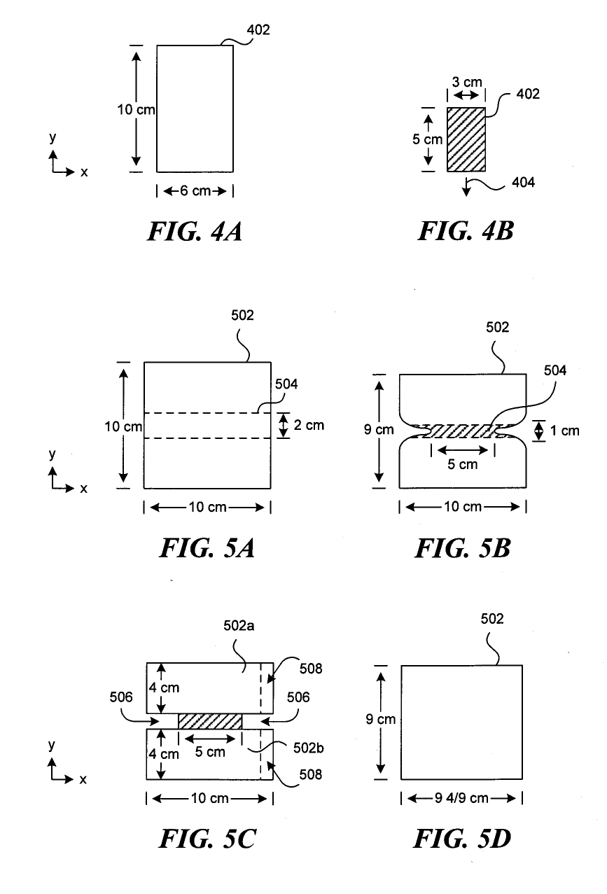

Certain embodiments of the disclosure orient the exposed regions of tissue with their longitudinal dimension transverse to the direction in which the greatest shrinkage or contraction is desired. This transverse orientation may include any non-parallel angle, including orienting the elongated regions normal to the contraction direction. The direction in which the greatest shrinkage is desired is typically parallel to a, stress (e.g., gravity) applied to the tissue. Orienting the elongated exposed regions transverse to an applied stress may seem counter intuitive as the individual exposed tissue regions will shrink more extensively transverse to the direction in which the overall contraction is desired. For example, it may seem that a longitudinal dimension of a rectangular geometry should be oriented parallel to the desired contraction direction. This conclusion may be correct if an entire contour zone of tissue is exposed to the energy. FIGS. 4A and 4B, for example, are schematic diagrams of a contour zone 402 illustrating the effect of exposing the entire contour zone 402 to energy to reduce the overall dimensions of the contour zone 402 by 50%. For example, FIG. 4A illustrates the contour zone 402 including representative dimensions of 10 cm by 6 cm before applying the energy. FIG. 4B illustrates the contour zone 4 02 after applying the energy (e.g., as shown with cross-hatching) and having reduced dimensions of 5 cm by 3 cm. This accordingly results in a greater net shrinkage in the y direction of 5 cm compared with a net shrinkage of 3 cm in the x direction.

However, if energy is applied to only an exposed region of the contour zone according to embodiments of the disclosure, rather than to the entire contour zone, the result changes. For example, FIGS. 5A-5D are a schematic diagrams illustrating the benefit of a contour zone 502 having an elongated exposed region 504 (shown in broken lines). Referring to FIGS. 5A-5D together, in FIG. 5A the contour zone 502 includes overall representative dimensions of 10 cm by 10 cm, and the exposed region 504 has representative dimensions of 2 cm by 10 cm. If sufficient energy is applied to the exposed region 504 to reduce each of its dimensions by 50%, the 2 cm dimension is reduced to 1 cm, resulting in an overall dimension of 9 cm in the y direction (see, e.g., FIG. 5B). The corresponding dimension in the x direction, however, varies with position along the y axis of the contour zone 502. For example, the dimension in the x-direction is 10 cm at the top and bottom portions of the contour zone 502, but only 5 cm in the center portion of the contour zone 502. A thorough analysis would show the x dimension of the exposed region 504 to curve smoothly along the y axis. Accordingly, the smooth curve may be approximated by rectangles, as illustrated in FIG. 5C, leaving gaps 506 proximate to the exposed region 504. The average dimension of the contour zone 502 in the x direction can be approximated by filling in the gap 506 with material adjacent to it, represented by side portions 508 (shown in broken lines). For example, FIG. 5D illustrates the typical or average dimension of the contour zone 502 in the x direction to be 9 4/9 cm when adjacent tissue of the side portions 508 is redistributed into the gap 506 (based on describing the average dimension of the contour zone 502 in the x direction by an integral average). As such, the tissue of the entire contour zone 502 shrinks overall by 1 cm in the y direction but only 5/9 cm in the x direction. Accordingly, exposing energy to an elongated exposed region 504 generally transverse to the direction in which shrinkage and contraction are desired (i.e., in the y-direction) in a contour zone 502 results in a greater amount of shrinkage in the y direction than in the x direction.

After applying energy according to embodiments of the disclosure, the subsequent application of an external stress will act first on the shrunk tissue (i.e., the exposed regions of tissue) to extend the contour zone. Accordingly, contour zones composed of exposed regions and non-exposed regions bear the preponderance of the stress while adjacent zones of non-exposed tissue bear a negligible portion of the stress. As such, exposing a greater overall cross section of tissue (e.g., the aggregate of the individual exposed regions) oriented transverse to the applied stress can be advantageous as this distributes the stress over the contour zone. This is particularly relevant if the exposure energy decreases the elastic modulus of the exposed tissue. Decreasing the elastic modulus of the exposed tissue results in an increased elongation of the tissue when a stress is subsequently applied to the tissue. Thus, tissue with broad exposed zones, rather than discrete exposed regions, is more likely to experience undesirable lengthening beyond the original, preshrunk length. Accordingly, the configuration of the exposed regions disclosed herein may be designed to avoid decreasing the elastic modulus of the contour zone and prevent undesirable lengthening of the tissue. For example, leaving regions of non-exposed tissue between the exposed regions can at least partially resist applied stresses by increasing the overall elastic modulus of the contour zone. Moreover, the geometric shapes described herein with aspect ratios greater than unity and with their longitudinal dimension oriented transversely to the applied stress are more effective in achieving this aim than the same geometric shape oriented parallel to the applied stress.

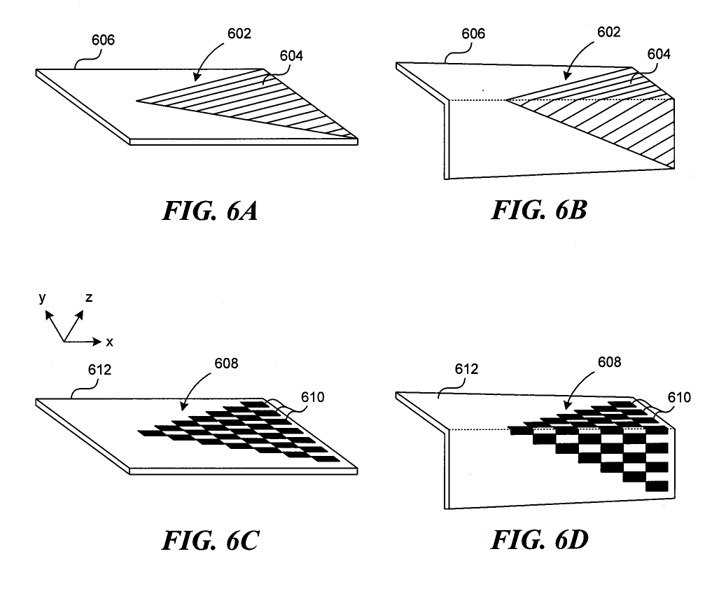

According to further embodiments of the disclosure, energy can be applied to the contour zones to contract the target tissue in three dimensions. For example, contour zones having a generally triangular shape or exposed regions arranged in a generally triangular pattern can achieve three-dimensional contouring of the tissue. FIGS. 6A and 6B, for example, are schematic diagrams of a contour zone 606 in accordance with embodiments of the disclosure. Referring to FIGS. 6A and 6B together, FIG. 6A illustrates the contour zone 606 before applying energy to a single exposed region 604 having a generally triangular shape 602. FIG. 6B illustrates the contour zone 606 after applying energy to the triangular exposed region 604. As illustrated in FIG. 6B, triangular shape of the contour zone 606 results in preferential contraction in three dimensions.

FIGS. 6C and 6D are schematic diagrams also illustrating a contour zone 612 configured to contract the target tissue in three dimensions. FIG. 6C illustrates the contour zone 612 before applying energy to a plurality of exposed regions 610 of tissue interspersed with unexposed regions configured in a generally triangular pattern 608. FIG. 6D illustrates the contour zone 612 after applying the energy to the plurality of exposed regions 610, such that the contour zone 612 is also preferentially shaped in three dimensions. The contour zones 606, 612 including generally triangular patterns 602, 608 of exposed regions 604, 610 may vary in number or magnitude across the tissue. The illustrated configurations are useful embodiments because they can allow for control of the curvature of the tissue in three dimensions with a two-dimensional exposure pattern.

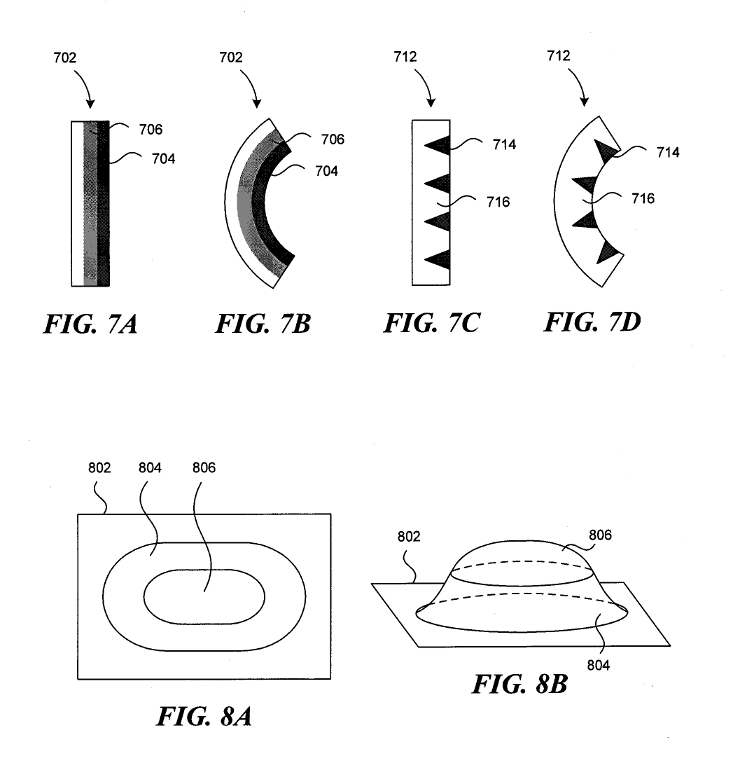

According to another embodiment of the disclosure, the depth of the energy exposure to the tissue may also be adjusted to induce a three-dimensional curvature of the target tissue. For example, the depth or intensity of exposure may differ in a single exposed region, or in one exposed region with reference to an adjacent exposed region. FIGS. 7A-7D are schematic side cross-sectional views of a target tissue having an induced curvature due to different depths of energy application. Referring first to FIGS. 7A and 7B, FIG. 7A represents a target tissue 702 having energy applied at different depths, and FIG. 7B represents the curved target tissue 702 after it has been preferentially contracted. In the illustrated embodiment, the energy is selectively applied to a first depth 704 and to a second depth 706 of the tissue 702. The selective amounts of energy applied to varying depths of the tissue 702 provide a net curvature of the tissue 702, as illustrated in FIG. 7B.

FIGS. 7C and 7D illustrate another embodiment of varying the depth of energy application to induce curvature in a target tissue 712. For example, FIG. 7C represents the energy exposure depths to the target tissue 712, and FIG. 7D represents the target tissue 712 after applying energy to contract the tissue. In the illustrated embodiment, energy is applied to a plurality of exposed regions 714 interspersed among non-exposed tissue 716. The energy penetrates the exposed regions 714 such that the depth of the exposure has a generally triangular shape. Thus, exposing target tissue at selectively varying depths can also contour the tissue into the desired direction and shape including, for example, a convex curvature with reference from inside the tissue. One skilled in the art will appreciate, however, that the present disclosure is not limited by the exposure depths of the illustrated embodiments. For example, energy may be applied to three or more depths or to exposure depths having shapes other than triangular shapes.

FIGS. 8A and 8B also illustrate the effect of varying energy exposure depth to contour tissue in three dimensions. More specifically, FIG. 8A is a top view of a contour zone 802 and FIG. 8B is an isometric view of the contour zone 802. Referring to FIGS. 8A and 8B together, the contour zone 802 includes a first exposure region 804 having energy applied to a first depth or intensity, and a second exposure region 806 having energy applied to a second depth or intensity. The first and second exposure regions 804, 806 can accordingly have concentric elongated regions to achieve the preferred contraction in three dimensions.

According to still further embodiments of the disclosure, energy can be applied in the manner described herein to at least partially induce auxetic properties or characteristics in the target tissues. Materials having auxetic properties generally become thicker in a direction perpendicular to an applied force. Stated differently, auxetic materials become thicker, not thinner, when stretched. Accordingly, in certain embodiments, the geometric shapes of the exposed regions of tissue can be specifically configured to induce auxetic characteristics in the target tissue. For example, the shape of an exposed region can include an ellipse, oval, isosceles triangle, triangle with rounded corners, reentrant square or cube, curved or squashed reentrant cube, fractal, laminate with multiple length scales, etc. Moreover, the pattern of the exposed regions can also be configured to induce auxetic characteristics in the tissue.

Applying energy to at least partially induce auxetic properties in tissue not otherwise displaying auxetic properties can help preserve the directionality of the shrinkage, even after the tissue is later subjected to an external stress. For example, preferentially shrinking a non-auxetic material along its longitudinal dimension followed by applying a stress to the same dimension will increase the length and reduce the width of the material, thereby directly countering the effect of the shrinkage. In effect, the applied stress decreases the aspect ratio of the exposed regions of tissue. However, applying the same stress to an auxetic material (e.g., tissue having induced auxetic characteristics from the geometrical exposure pattern) increases both the length and width of the material, thus preserving, at least partially, the directionality introduced during shrinkage. Another advantage of inducing auxetic properties in the target tissue is that the auxetic properties can help contour the tissue in three dimensions. For example, materials having auxetic properties naturally adopt a synclastic curvature. Accordingly, tissue having induced auxetic properties can enhance the three-dimensional contouring effect (e.g., contouring the tissue around the jaw).

### C. Embodiments of Tissue Contouring Apparatuses, Devices and Systems

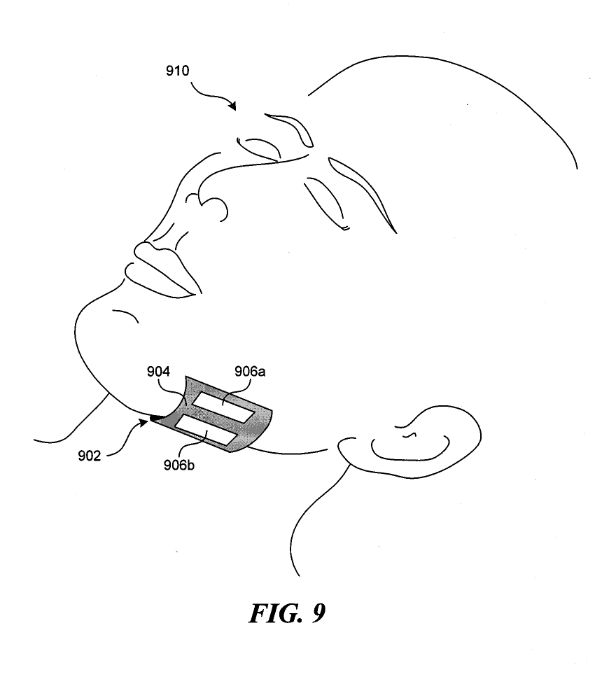

Several of the embodiments of methods of contouring and shaping tissue described herein can be applied with various devices, apparatuses and systems. These devices and methods can be configured to deliver various forms of energy to the tissue transcutaneously, transluminally, transendoscopically and/or through incisions. In one embodiment, for example, an apparatus can be positioned proximate to the target tissue to facilitate the energy delivery to the exposure regions having the desired geometry and/or pattern. FIG. 9, for example, is an isometric view of an apparatus including a template 902 positioned proximate to a face 910 provide a non-invasive face lift (or other type of tissue contouring), according to an embodiment of the disclosure. The template 902 can be removably positioned or applied to a portion of tissue of the face 910 (e.g., the brow, cheek, neck, jowl, etc.). The template 902 includes a body 904 having one or more openings or windows 906 (identified individually as first and second windows 906a, 906b) to allow the energy to be transmitted primarily through the windows 906 to achieve the directional shrinkage. For example, in certain embodiments the windows 906 define the exposed regions of tissue as described above. Accordingly, an energy applicator (not shown) can be placed on the template 902 such that the only area of tissue exposed to the applied energy is that under the corresponding window(s) 906. In certain embodiments, the body 904 of the template 902 can be made of a flexible non-conductive material having an adhesive on the side contacting the skin such that the template 902 can be applied to the tissue and generally conform to the tissue. For example, the template 902 can be of specific shape and size to fit the anatomical needs of the procedure, such as around the jaw for the reshaping of the jowl line. In other embodiments, however, the body 904 can be made from other materials, including non-flexible materials.

In certain embodiments, an energy applicator can apply the energy directly to the tissue through the windows 906. For example, the template 902 can be placed on a contour zone of tissue with the windows 906 oriented in a preferred direction, and the energy applicator can be positioned on the template 902 to directly contact the tissue exposed through the windows 906. In other embodiments, however, the template 902 can allow the energy applicator to indirectly apply energy to the target tissue. For example, the template 902 can include different members or materials in the windows 906 to selectively allow certain energies to pass to the target tissue. The template 902 can also be spaced away from the target tissue such that the energy is applied through the windows 906. In still further embodiments, the template 902 can be attached or otherwise positioned on the energy applicator to provide the desired energy exposure to the tissue.

Although the illustrated template 902 includes two windows 906, in other embodiments the template 902 can include any number of windows 906, including a single window 906. Moreover, although the illustrated windows 906 have a generally rectangular geometry, the windows 906 can be configured to have any of the geometric shapes described above, including, for example, shapes with an aspect ratio exceeding unity and/or in the shape of an ellipse, oval, rectangle, rectangle with rounded corners, isosceles triangle, triangle with rounded corners, reentrant square or cube, curved or squashed reentrant cube, fractal, laminate with multiple length scales etc. The windows 906 can also have different sizes and be positioned in different orientations or patterns according to the embodiments discussed above. For example, the windows 906 can be arranged in columns, staggered, rotated at an angle relative to adjacent windows 906, etc. Moreover, in certain embodiments, the windows 906 can include a transparent or partially opaque member to allow selective transmission of energy. In still further embodiments, the windows 906 can include a thermally insulating material that is transparent to applied energy. Moreover, one skilled in the art will appreciate that although the illustrated template 902 is applied to the face 910, in other embodiments, a template can be used for several different clinical applications, such as a brow lift, paravaginal tissue shrinkage to treat stress urinary incontinence, a breast lift, etc.

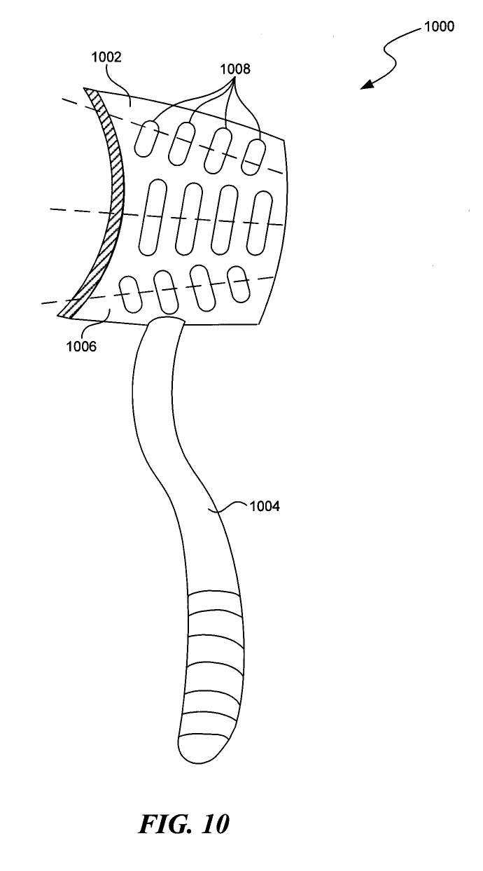

According to another embodiment of the disclosure, an apparatus for facilitating the selective application of energy to tissue can include a probe having a template. FIG. 10, for example, is an isometric view of a probe 1000 including a template 1002 coupled to a handle 1004. The template 1002 can include several features that are generally similar to the template 902 described above with reference to FIG. 9. For example, the template 1002 includes a body 1006 having a plurality of windows 1008 that can include any of the shapes or geometries arranged in different patterns as described above. Although the illustrated windows 1008 are generally arranged in columns, in certain embodiments the columns may not be parallel to each other to enable shrinkage in more than one direction (as illustrated in FIG. 10). In other embodiments, however, the pattern of the windows 1008 can vary according to the desired tissue shaping. Supporting the template 1002 with the handle 1004 allows a user to quickly reposition the template 1004 for different shrinkage directions between energy applications. For example, the probe 1000 can be moved to treat the different areas in a predetermined sequence by the user to achieve the desire tissue contouring effect.

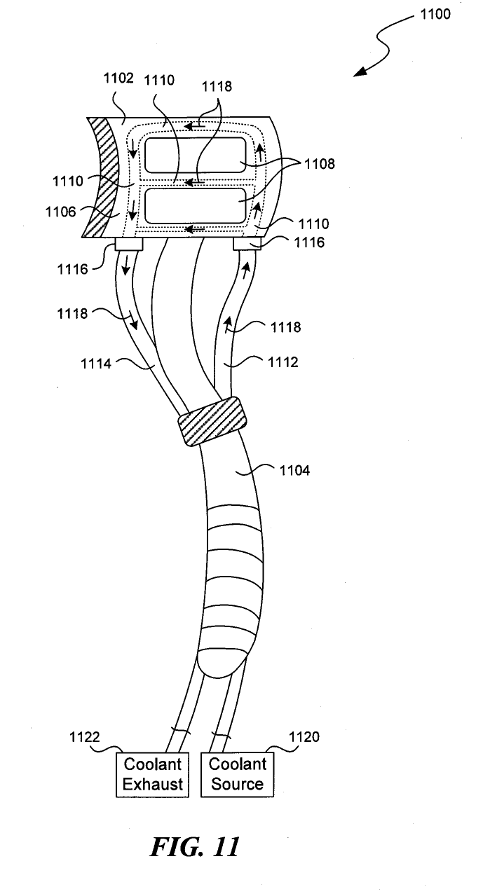

FIG. 11 is an isometric view of a cryoprobe 1100 configured in accordance with another embodiment of the disclosure. The illustrated cryoprobe 1100 includes a template 1102 coupled to a handle 1104. The template 1102 includes a body 1106 having one or more windows 1108 to allow energy to pass to the target tissue in a specified configuration, similar to the embodiments described above with reference to FIGS. 9 and 10. In this embodiment, however, the body 1106 is configured to cool tissue volumes surrounding the target tissue exposed by the windows 1108. In a specific embodiment, the body 1106 is configured to circulate an internal coolant. As illustrated in FIG. 11, for example, the body 1106 includes a plurality of internal channels 1110 (shown in broken lines) and the probe 1100 includes a first conduit 1112 for introducing the coolant into the channels 1110 and a second conduit 1114 for removing the coolant from the channels 1110. Each of the first and second conduits 1112, 1114 are coupled to the body 1106 with connectors 1116 to allow the coolant to flow through the channels 1110, as shown by a plurality of arrows 1118 illustrating the coolant flow. In addition, the first conduit 1112 can be coupled to a coolant source 1120, and the second conduit 1114 can be coupled to a coolant exhaust 1122 or to the coolant source 1120 to recycle the coolant. The coolant can be composed of any substance suitable for cooling the body 1106, and can flow through the probe 1100 in a liquid or gas form. Moreover, in certain embodiments, the windows 1108 can be open or they can include thermally insulated material to prevent the target tissue under the windows 1108 from being cooled. For example, the insulated material can be trapped air or composed of thermally nonconductive polymers.

In other embodiments, the cryoprobe 1100 may not include a coolant flowing through the body 1106. Rather, at least a portion of the template 1102 can be otherwise cooled before using the template 1102 to cool the tissue surrounding the target tissue. For example, in a specific embodiment, the template 1102 can be submerged in a cooling medium (e.g., water) prior to applying the energy with the template. In another embodiment, the body 1106 can be cooled by electrical means. For example, the body 1106 can include a thermoelectric unit configured to provide thermoelectric cooling to portions of the target tissue. In the embodiments described above with reference to cooling probes, the templates can cool the tissue surrounding the target tissue before, during and/or after the application of the energy to the target tissue.

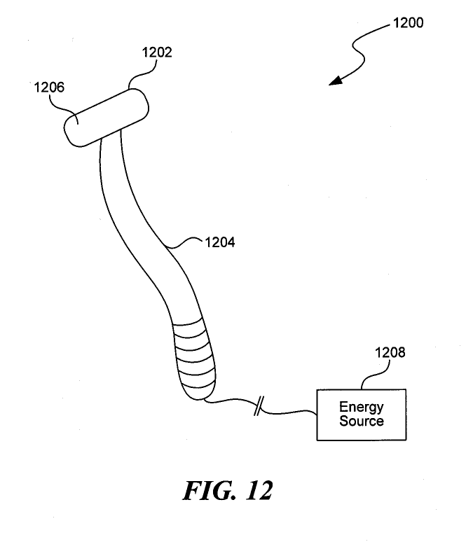

Another embodiment of the disclosure is directed to a probe configured to deliver energy directly to the target tissue. This embodiment differs from the previous embodiments in that the energy is applied to the tissue from a contact surface of the probe, rather than applying energy through a window or template. FIG. 12, for example, is an isometric view of a probe 1200 including a body or applicator head 1202 coupled to a handle 1204. In certain embodiments, the head 1202 is configured to include any the geometric shapes described above to form a contact surface 1206 to apply the energy to the target tissue. The illustrated contact surface 1206, for example, has an aspect ratio exceeding unity. In a specific embodiment, the contact surface 1206 has a footprint including dimensions of about 1 cm by 2 cm to shrink the tissue to a greater extent more selectively in a direction parallel to the 1 cm dimension. In other embodiments, however, the contact surface 1206 can have other dimensions or configurations. For example, the contact surface 1206 can include a plurality of spaced apart energy applying surfaces to create a pattern of exposed regions of tissue as described above.

The probe 1200 can be operatively coupled to an energy source 1208 to allow the head 1202 to deliver energy to the tissue. In certain embodiments, for example, the probe 1200 is a radiofrequency (RF) probe (e.g., a monopolar RF probe) coupled to an RF source. The RF probe can accordingly heat the target tissue by the thermionic effect of the applied RF energy. In other embodiments, the probe can deliver other types of energy to the target tissue. For example, the contact surface 1206 can be configured to heat the target tissue by conduction. In further embodiments the probe 1200 can be coupled to a laser source and configured to deliver a focused laser beam to penetrate and heat the subcutaneous tissue. In these embodiments the probe can direct the focused laser beam to rapidly expose an area of tissue, with the aspect ratio of the tissue area exceeding unity. The energy applying probes disclosed herein accordingly allow a user to easily and quickly apply energy to a target tissue at different angles or orientations to tighten and contour the tissue as desired by the user.

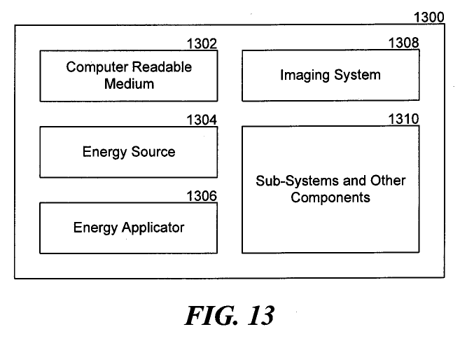

FIG. 13 is a schematic diagram of a system 1300 configured in accordance with an embodiment of the disclosure. The system can include a computer readable medium 1302, an energy source 1304, an energy applicator 1306, an imaging system 1308 and/or other subsystems or components 1310. The components of the system 1300 can be operably coupled as a single unit or distributed over multiple interconnected units (e.g., through a communication network). In certain embodiments, the computer readable medium 1302 contains instructions that cause the energy applicator 1306 to selectively apply energy to a contour zone of tissue to contour the tissue in a predetermined direction. As such, a clinician can apply the energy to discrete portions of tissue in the contour zone according to the embodiments disclosed herein. The imaging system 1308 can produce an image of at least a portion of the contour zone, including the subcutaneous tissue of the contour zone, before, during and after the energy exposure. The imaging system 1308 can include, for example, x-ray, ultrasound, CT scan, positron emission topography (PET), MRI, etc. The imaging system 1308 can accordingly identify the fascial and supporting tissue layers of the target tissue in all three dimensions before, during and after the energy application.

### D. Applications of the Disclosed Tissue Contouring Methods and Apparatuses

The methods and devices disclosed herein can be used for many different applications. One application of the embodiments described herein, for example, is the mastopexy as described above with reference to FIGS. 2A-2C. The disclosed procedures accordingly achieve a breast lift and contouring comparable to open surgery without the complicated and invasive steps required for conventional breast lifts.

A face lift is another example where the disclosed methods and apparatuses of directional tissue shrinkage can be used to accomplish contour remodeling. The objective of a face lift, which is a surgical procedure conventionally performed by either open or endoscopic techniques, is to tighten and to rebalance the subcutaneous musculoaponeurotic system (SMAS) in specific directions over different zones of the forehead, face and/or neck. With conventional procedures, imparting directionality to the SMAS is generally accomplished by cutting and suturing the tissue in strategic areas along specific directions. Tightening the skin by suturing enables a surgeon to remodel different zones of the forehead, face and neck to reverse the sagging or loosening of facial tissue caused by gravity and the aging process, resulting in a more youthful appearance.

Although conventional face lift techniques may achieve the desired contour of the skin, these techniques are generally invasive. For example, the cutting and suturing involved in a conventional face lift may require extended recovery time and increase the risk of infection. In addition, scars are often visible after the surgery. Moreover, arbitrarily applying energy to the SMAS will not likely contour or shape the tissue according to precise or desired results. According to the embodiments described herein, however, these limitations can be overcome. For example, by applying energy in a specific geometry and/or pattern to the SMAS, the tissue in the different zones of the forehead, face and neck can be tightened in specific directions in a non-invasive manner with results similar to what a surgeon would achieve by cutting and suturing during a surgical face lift.

The foregoing examples of breast and face lifts are specific embodiments of clinical applications that benefit from the non-invasive tissue shaping techniques disclosed herein. There are, however, many other cosmetic applications that can be used to treat conditions where shrinkage of collagen containing tissues or regions of collagen containing tissues and other tissue types has a therapeutic effect. Other cosmetic applications include, for example, brow and neck lefts, arm lifts, abdominoplasty, buttock or thigh lift, calf contouring, genital plastic surgery, etc. The disclosed embodiments can also be used for genital-urinary system applications. For example, the disclosed techniques and apparatuses can be used for treating stress urinary incontinence, genital prolapse or for vaginal tightening.

From the foregoing, it will be appreciated that specific embodiments of the disclosure have been described herein for purposes of illustration, but that various modifications may be made without deviating from the spirit and scope of the disclosure. Aspects described in the context of particular embodiments may be combined or eliminated with other embodiments. Further, although advantages associated with certain embodiments have been described in the context of those embodiments, other embodiments may also exhibit such advantages, and not all embodiments need necessarily exhibit such advantages to fall within the scope of the disclosure. Accordingly, the disclosure is not limited except as by the appended claims.

I/we claim

1. A method for contouring tissue, the method comprising:
determining a contraction direction along which the tissue is to be contracted to a greater extent than other directions; and
applying energy to a plurality of discrete elongated exposed regions of tissue spaced apart from each other among non-exposed regions of tissue, wherein the exposed regions are oriented such that a longitudinal dimension of at least one of the exposed regions is generally transverse to the contraction direction.

2. The method of claim 1 wherein determining the contraction direction includes determining the contraction direction to be generally parallel to an external applied stress.

3. The method of claim 2 wherein the external applied stress comprises gravity.

4. The method of claim 1 wherein determining the contraction direction comprises determining a first contour zone, the method further comprising determining a second contraction direction defined by a second contour zone spaced apart from the first contour zone, and wherein applying energy to the plurality of elongated exposed regions comprises forming a plurality of first elongated exposed regions in the first contour zone, each of the first elongated exposed regions being oriented generally transverse to the first contraction direction, and forming a plurality of second elongated exposed regions in the second contour zone, each of the second elongated exposed regions being oriented generally transverse to the second contraction direction.

5. The method of claim 4 wherein forming the plurality of first elongated exposed regions in the first contour zone comprises at least partially staggering the first elongated exposed regions relative to each other.

6. The method of claim 4 wherein forming the plurality of first elongated exposed regions in the first contour zone comprises non-uniformly distributing the exposed regions throughout the first contour zone.

7. The method of claim 4 wherein forming the plurality of first elongated exposed regions in the first contour zone comprises creating a first density of elongated exposed regions in a first area of the first contour zone and a second density of elongated exposed regions in a second area of the first contour zone.

8. The method of claim 1 wherein the elongated exposed regions comprise at least one elongated exposed region having a geometry different from a geometry of another of the plurality of elongated exposed regions.

9. The method of claim 1 wherein at least one of the elongated exposed regions has an aspect ratio that is greater than unity.

10. The method of claim 1 wherein applying energy to the plurality of discrete elongated exposed regions comprises at least partially inducing auxetic properties in the tissue.

11. The method of claim 1 wherein applying energy to a plurality of discrete elongated exposed regions includes applying energy to one or more exposed regions comprised of a shapes including at least one of an ellipse, oval, rectangle, rectangle, isosceles triangle, triangle with generally rounded corners, reentrant square, reentrant cube, fractals, and laminate with multiple length scales.

12. The method of claim 1 wherein at least one of the elongated exposed regions comprises a geometry having generally rounded corners.

13. The method of claim 1 wherein applying energy comprises applying at least one of visible light, infrared light, ultraviolet light, radio frequency, microwave, ultrasound, direct heat and high intensity focused ultrasound.

14. The method of claim 1 wherein applying energy comprises directing energy to the elongated exposed regions of tissue from an energy applying device in direct contact with the tissue.

15. The method of claim 1 wherein applying energy comprises directing energy to the elongated exposed regions of the tissue from an energy applying device spaced apart from the tissue.

16. The method of claim 1, further comprising guiding the energy application by imaging the elongated exposed regions with one or more of direct visualization, direct visualization with magnification, microphotography, digital image processing, analog image processing, infrared imaging, radiography, computer tomography, magnetic resonance imaging, ultrasound and positron emission tomography.

17. A method for contouring tissue, the method comprising:
determining an arrangement of at least one contour zone of tissue; and
selectively applying energy to one or more discrete portions of the tissue in the contour zone, wherein selectively applying the energy contracts at least a portion of the tissue in a predetermined direction to a greater extent relative to directions at an angle to the predetermined direction.

18. The method of claim 17 wherein:
determining an arrangement comprises:
determining a predetermined contraction direction of the tissue in the contour zone along which the tissue is to be contracted to a greater extent than other directions; and
forming at least one elongated exposed region of tissue in the contour zone, wherein a longitudinal dimension of the exposed region is positioned generally transverse to the contraction direction; and
selectively applying energy comprises directing the energy to the exposed region to at least partially heat the tissue.

19. The method of claim 17 wherein selectively applying energy comprises at least partially shrinking the tissue.

20. The method of claim 17 wherein selectively applying energy comprises contouring the tissue in three dimensions.

21. The method of claim 20 wherein contouring the tissue in three dimensions comprises applying the energy to at least two different depths in the tissue.

22. The method of claim 17 wherein applying energy comprises sequentially applying energy to a first portion of tissue and to a second portion of tissue spaced apart from the first portion in the contour zone.

23. The method of claim 17 wherein applying energy comprises simultaneously applying energy to a first portion of tissue and to a second portion of tissue spaced apart from the first portion in the contour zone.

24. The method of claim 17, further comprising cooling an area of the tissue at least partially surrounding the portion of tissue that the energy is selectively applied to.

25. An apparatus for shaping tissue, the apparatus comprising:
a support member; and
an energy applicator coupled to the support member, wherein the energy applicator has a longitudinal dimension and a lateral dimension less than the longitudinal dimension such that the energy applicator is configured to apply energy to an area of the tissue to contract the tissue in a predetermined direction.

26. The apparatus of claim 25 wherein the applicator is configured to apply the energy to the area of the tissue such that the tissue contracts a greater amount in the predetermined direction than in a second direction generally transverse to the predetermined direction.

27. The apparatus of claim 25 wherein the applicator comprises a contact surface for contacting the area of the tissue to apply the energy to the tissue, wherein the contact surface has a predetermined shape including an aspect ratio that exceeds unity.

28. The apparatus of 27 wherein the shape of the contact surface is a rectangular shape having generally rounded corners.

29. The apparatus of claim 25 wherein the applicator is configured to apply energy directly to a surface of the tissue to increase a temperature of a portion of subcutaneous tissue below the tissue surface by conduction.

30. The apparatus of claim 25 wherein the applicator is configured to apply energy in the form of at least one of radio frequency electric energy, infrared radiation, visible light radiation, ultraviolet radiation, microwave radiation, laser energy, maser energy, ultrasound energy and high intensity focused ultrasound energy.

31. An apparatus for facilitating tissue contouring, the apparatus comprising:
a body configured to be positioned proximate to the tissue; and
at least one exposure area in the body, wherein the exposure area has a longitudinal dimension and a lateral dimension, the longitudinal dimension being greater than the lateral dimension, and wherein the exposure area exposes a target area of the tissue such that an energy can be applied through the exposure area to the target area.

32. The apparatus of claim 31 wherein the exposure area exposes the target area such that energy can be applied through the exposure area to contract the tissue in a predetermined direction generally transverse to the longitudinal dimension of the exposure area.

33. The apparatus of claim 31 wherein the exposure area comprises an opening in the body having a rectangular geometry with generally rounded corners.

34. The apparatus of claim 31, further comprising a member positioned in the exposure area, wherein the member is composed of a transparent material configured to allow a selective transmission of the energy applied to the tissue through the exposure area.

35. The apparatus of claim 31 wherein the apparatus is a portable apparatus configured to be repositioned at more than one position proximate to the tissue, and wherein the body is configured to be placed proximate to the tissue such that the longitudinal dimension of the exposure area can be positioned generally transverse to an external stress applied to the tissue.

36. The apparatus of claim 31 wherein the exposure area comprises an opening in the body to expose the target area so that the energy can be applied directly to the target area through the opening.

37. The apparatus of claim 31 wherein the exposure area comprises an opening in the body to expose the target area so that the energy can be applied indirectly to the target area through the opening.

38. The apparatus of claim 31, further comprising a support member attached to the body, wherein the support member is configured to allow a user to position and orient the body at different positions proximate to the tissue.

39. The apparatus of claim 31 wherein the exposure window is a first exposure window, the apparatus further comprising a plurality of exposure windows arranged in a predetermined array, wherein each of the exposure windows includes a longitudinal dimension and a lateral dimension, the longitudinal dimension being greater than the lateral dimension.

40. The apparatus of claim 31 wherein the body comprises a first side proximate to the tissue and a second side opposite the first side, the apparatus further comprising an adhesive at the first side of the body to facilitate temporary attachment of the body to the tissue.

41. The apparatus of claim 31, further comprising:
a support member attached to the body, wherein the support member is configured to allow a user to reposition the body proximate to the tissue; and
at least one channel extending through the body through which a coolant can be disposed, wherein the channel extends through a portion of the body that is proximate to the exposure area.

42. The apparatus of claim 31 wherein the body comprises a thermoelectric unit configured to selectively cool portions of the tissue.

43. The apparatus of claim 31 wherein the exposure area is configured to expose the target area to at least one of radio frequency electric energy, infrared radiation, visible light radiation, ultraviolet radiation, microwave radiation, laser energy, maser energy, ultrasound energy and high intensity focused ultrasound energy.

44. A system for contouring tissue, the system comprising:
a computer readable medium;
an energy source operably coupled to the computer readable medium; and
an energy applicator operably coupled to the energy source, wherein the computer readable medium contains instructions that cause the applicator to selectively apply energy to shrink at least a portion of the tissue in a predetermined direction.

45. The system of claim 44, further comprising an imaging system operably coupled to the computer readable medium and configured to produce an image of at least a portion of a contour zone of the tissue.

46. The system of 45 wherein the imaging system includes one or more of an x-ray, ultrasound, CT scan, positron emission topography and MRI system.

47. The system of claim 44 wherein instructions further cause the applicator to selectively apply the energy to a plurality of exposure regions of the tissue arranged in a predetermined array on the tissue, wherein individual exposure regions include a geometry having an aspect ratio greater than one and wherein at least one of the exposure regions has a longitudinal dimension that is oriented in a direction generally transverse to the predetermined direction.
